(19) United States
(12) Patent Application Publication
(10) Pub. No.: US 2016/0040214 A1
(43) Pub. Date: Feb. 11, 2016

(54) Nucleic Acid Quantitation Method

(71) Applicant: Accugen Pty Ltd., Roseberry (AU)

(72) Inventors: Karen A. Duggan, Clovelly (AU); Nicola R. Boulter, Beecroft (AU)

(21) Appl. No.: 14/885,484

(22) Filed: Oct. 16, 2015

Related U.S. Application Data
(63) Continuation-in-part of application No. 13/393,763, filed on May 21, 2012.
(60) Provisional application No. 62/065,455, filed on Oct. 17, 2014.

(30) Foreign Application Priority Data
Sep. 2, 2009 (AU) 2009904258

(51) Int. Cl. C12Q 1/68

(52) U.S. Cl. CPC C12Q 1/686

(57) ABSTRACT

The present method relates to methods of quantifying nucleic acids, and in particular to the use of a universal reference nucleic acid to generate a calibration curve from which the level of a target nucleic acid in a sample can be calculated.

### FIELD OF THE INVENTION

The present invention relates to methods of quantifying nucleic acid without the need for normalising data to a reference gene or synthetic gene of interest. In particular, the invention relates to an improved universal method of quantifying nucleic adds for gene expression studies. This method is applicable to diagnostic, forensic and research use. However, it will be appreciated that the invention is not limited to these particular fields of use.

### BACKGROUND OF THE INVENTION

Any discussion of the prior art throughout the specification should in no way be considered as an admission that such prior art is widely known or forms part of the common general knowledge in the field.

PCR technologies for quantification of gene expression have improved through the development of rapid thermocyclers and the introduction of fluorescence monitoring of amplified products after each cycle (real-time PCR). Quantification of gene expression occurs through the use of dyes, particularly fluorescent dyes, and the detection of increasing fluorescence during the exponential phase of PCR amplification proportional to the amount of nucleic acids in the sample at the beginning of the reaction. Quantification is based on the threshold cycle, i.e. the first cycle with detectable fluorescence, and can be performed in an absolute manner with external standards (usually a synthetic gene) or in relative manner with a comparative normalizing reference gene serving as an internal calibrator (i.e. reference gene). Control genes or reference genes are used to normalise mRNA levels between different samples.

It is critical that the selected reference gene does not fluctuate since variations in gene expression will alter the relative quantification profile of the target gene. Pipetting and dilution errors also alter the level of amplification and thus alter the quantification profile.

Genes such as glyceraldehyde-3-phosphate dehydrogenase (GAPDH), porphobilinogen deaminase (PBGD), beta2-microglobin (B2M) or beta-actin (Actb) are often used as internal calibrators in real-time PCR. However, the aforementioned genes have been shown to change expression levels in response to experimental conditions or treatments. Genes that are abundantly expressed, such as 18S, are also not ideal reference genes as PCR conditions need to be restricted so as not to swamp the reaction.

Thus, suitable reference genes should be adequately expressed in the tissue of interest, and most importantly, show minimal variability in expression between the samples and under the experimental conditions or treatments used.

Many of these control genes however can show unacceptable variability in expression. It has been shown that the expression level of such genes may vary among tissues or cells and may change under certain circumstances i.e. different treatments. Thus it is crucial to validate reference genes in any new experimental system. It is often a time consuming and difficult task to find a reference gene that is suitable for use in a specific experimental system, in some situations this may not be possible.

The use of external standards (i.e. a synthetic reference) in gene expression studies generally requires that the gene of interest be cloned to provide the synthetic reference gene. In this method, known amounts of the synthetic reference gene sequence are serially diluted then subjected to amplification to produce a standard curve. Production of the cloned sequence for this method is generally a time consuming, labour intensive task and dilution errors are amplified exponentially which can lead to inaccurate assessment of nucleic acid levels. Stability and preservation of highly diluted cloned sequences can also cause difficulties.

Thus, there remains a need for a simple and efficient universal method of quantifying nucleic acids that is applicable to any experimental situation or treatment condition that does not require the use of a reference gene or synthetic gene of interest to normalise data and/or quantify gene expression.

It is an object of the present invention to overcome or ameliorate at least one of the disadvantages of the prior art, or to provide a useful alternative.

### SUMMARY OF THE INVENTION

In a broad aspect, the present invention relates to a method for quantifying nucleic acids of interest that does not require the use of a reference gene or synthetic gene of interest to normalize nucleic acid expression data. Rather the method of the present invention utilizes a universal reference nucleic acid which can be used to generate a calibration curve from which the level of an amplified target nucleic acid in a sample can be calculated. The present invention also relates to kits for use in the method of the invention.

In a first aspect, the present invention provides a method for quantifying a target nucleic acid, the method comprising the steps of:

measuring the fluorescence of known quantities of a fluorophore-tagged universal reference nucleic acid to generate a calibration curve;

amplifying the target nucleic acid in the presence of a fluorophore-tagged probe, wherein the fluorophore-tagged probe hybridizes with the target nucleic acid:

measuring fluorescence during amplification of the target nucleic acid:

comparing fluorescence during amplification with the calibration curve and determining the quantity of the amplified target nucleic acid.

In one embodiment, steps (a) to (d) are conducted substantially simultaneously.

In another embodiment, the fluorophore-tagged universal reference nucleic acid is single stranded.

In another embodiment, the calibration curve is generated using from about 0 nM to about 500 nM of fluorophore-tagged universal reference nucleic acid.

In another embodiment, the calibration curve is generated using from about 0 nM to about 200 nM of fluorophore-tagged universal reference nucleic acid.

In another embodiment, the calibration curve is generated using at least three known quantities of the fluorophore-tagged universal reference nucleic acid.

In another embodiment, the calibration curve is generated using at least six known quantities of the fluorophore-tagged universal reference nucleic acid.

In another embodiment, the calibration curve is generated using about 0 nM, about 20 nM, about 40 nM, about 60 nM, about 80 nM, about 100 nM and about 120 nM of the fluorophore-tagged universal reference nucleic acid.

In another embodiment, the calibration curve is generated using about 0 nM, about 20 nM, about 40 nM, about 60 nM, about 80 nM, about 100 nM, about 120 nM, about 140 nM, about 160 nM and about 200 nM of the fluorophore-tagged universal reference nucleic acid.

In another embodiment, the universal reference nucleic acid has a length of less than about 60 bp.

In another embodiment, the universal reference nucleic acid has a length of greater than about 170 bp.

In another embodiment, the universal reference nucleic acid has a length of about 20 bp.

In another embodiment, the universal reference nucleic acid has a GC content of less than about 45%, preferably less than about 40%, preferably less than about 35%, preferably less than about 30%, preferably less than about 25%, preferably less than about 20%, preferably less than about 15%, preferably less than about 10%.

In another embodiment, the universal reference nucleic acid has a GC content of greater than about 60%, preferably greater than about 65%, preferably greater than about 70%, preferably greater than about 75%, preferably greater than about 80%, preferably greater than about 85%, preferably greater than about 90%.

In another embodiment, the universal reference nucleic acid has a GC content of about 30%.

In another embodiment, the universal reference nucleic acid has a GC content of about 40%.

In another embodiment, the universal reference nucleic acid has a GC content of about 60%.

In another embodiment, the universal reference nucleic add has a GC content of about 80%. In another embodiment, the universal reference nucleic acid has a length of about 21 bp and a GC content of about 62%.

In another embodiment, the target nucleic acid has a length of less than about 60 bp.

In another embodiment, the target nucleic acid has a length of greater than about 150 bp.

In another embodiment, the target nucleic acid has a length of greater than about 210 bp.

In another embodiment, the target nucleic acid has a length of about 90 bp.

In another embodiment, the target nucleic acid has a length of about 500 bp.

In another embodiment, the target nucleic acid has a length of about 1000 bp.

In another embodiment, the target nucleic acid has a GC content of about 75%.

In another embodiment, the target nucleic acid has a GC content of about 25%. In another embodiment, amplification of the target nucleic acid is by polymerase chain reaction (PCR).

In another embodiment, the PCR is real-time PCR.

In another embodiment, the real-time PCR is multiplex PCR.

In another embodiment, the fluorophore-tagged universal reference nucleic acid and the fluorophore-tagged probe are detectable in the same emission channel.

In another embodiment, the fluorophore-tagged universal reference nucleic acid and the fluorophore-tagged probe have the same excitation and emission spectra.

In another embodiment the fluorophore is selected from the group consisting of FAM, HEX, Texas Red, TYE665, Alexa 594 and Cy5.

In another embodiment, the PCR is performed over about 30 to about 45 cycles.

In a second aspect, the present invention provides a kit comprising:

(a) one or more fluorophore-tagged universal reference nucleic acids;
(b) one or more fluorophore-tagged probes; and
(c) instructions for use in the method of the first aspect.

In a third aspect, the present invention provides a kit when used in the method of the first aspect, the kit comprising:

(a) one or more fluorophore-tagged universal reference nucleic acids; and
(b) one or more fluorophore-tagged probes.

The universal reference nucleic acid sequence need not have any homology with the target nucleic acid or with any reference gene sequence. However, because of the particular way in which the universal reference nucleic acid is used in the method of the present invention (i.e. to prepare a calibration curve following serial dilution of the universal reference nucleic acid) the universal reference nucleic acid sequence can have a degree of homology or can even be identical with a target nucleic acid sequence or a reference gene, or smaller parts thereof.

An advantage of the present invention is that the method can make use of the same universal reference nucleic acid to quantify different target nucleic acids.

The universal reference nucleic acid may be of the same or similar length to the target nucleic acid sequence but this need not be so. The universal reference nucleic acid may be longer or shorter than the target nucleic acid. The universal reference nucleic acid may be double or single stranded. The universal reference nucleic acid may be obtained from a biological source, natural or otherwise, using known techniques, or it may be prepared synthetically.

The universal reference nucleic acid and the probe are both tagged with a fluorophore. Suitable fluorophores would be known to the skilled addressee and include, but are not limited to, FAM, JOE, HEX, Alexa 594, Texas Red, Cy5 and TYE665. When used in real time PCR, the fluorophores should be selected so that they are detectable in the emission channels of the real time PCR device.

Preferably a single calibration curve is prepared with the universal reference nucleic acid and used for multiple target nucleic acid amplifications and quantifications.

Preferably, the amplification of the target nucleic add is performed by Polymerase Chain Reaction (PCR) method. Typically the target nucleic acid is amplified over 30 to 40 cycles but this is not critical to the method of the present invention. Simultaneous amplification of multiple target nucleic acids of interest in one reaction can also be performed (i.e., multiplex PCR).

Unless the context clearly requires otherwise, throughout the description and the claims, the words “comprise”, “comprising”, and the like are to be construed in an inclusive sense as opposed to an exclusive or exhaustive sense, that is to say, in the sense of “including, but not limited to”.

### BRIEF DESCRIPTION OF THE FIGURES

FIG. 1: Fluorescence measured over 40 PCR cycles for a FAM-tagged reference nucleic acid of 21 bp and 30% GC content, diluted to 0 nM, 20 nM, 40 nM, 60 nM, 80 nM, 100 nM and 120 nM (FIG. 1A). Calibration curve created by plotting the fluorescence value at each cycle against the concentration of reference nucleic acid and the linear regression was calculated (FIG. 1B). Fluorescence measured over 40 PCR cycles during amplification of a 92 bp fragment from serial dilutions of a stock lambda DNA using a FAM-tagged hydrolysis probe (FIG. 1C).

FIG. 2: Fluorescence measured over 40 PCR cycles for a FAM-tagged reference nucleic acid of 21 bp and 40% GC content, diluted to 0 nM, 20 nM, 40 nM, 60 nM, 80 nM, 100 nM and 120 nM (FIG. 2A). Calibration curve created by plotting the fluorescence value at each cycle against the concentration of reference nucleic acid and the linear regression was calculated (FIG. 2B). Fluorescence measured over 40 PCR cycles during amplification of a 92 bp fragment from serial dilutions of a stock lambda DNA using a FAM-tagged hydrolysis probe (FIG. 2C).

FIG. 3: Fluorescence measured over 40 PCR cycles for a FAM-tagged reference nucleic acid of 21 bp and 62% GC content, diluted to 0 nM, 20 nM, 40 nM, 60 nM, 80 nM, 100 nM, 120 nM, 140 nM, 160 nM and 200 nM and (FIG. 3A). Calibration curve created by plotting the fluorescence value at each cycle against the concentration of reference nucleic acid and the linear regression was calculated (FIG. 3B). Fluorescence measured over 40 PCR cycles during amplification of a 92 bp fragment from serial dilutions of a stock lambda DNA using a FAM-tagged hydrolysis probe (FIG. 3C).

FIG. 4: Fluorescence measured over 40 PCR cycles for a FAM-tagged reference nucleic acid of 21 bp and 80% GC content, diluted to 0 nM, 20 nM, 40 nM, 60 nM, 80 nM, 100 nM and 120 nM (FIG. 4A). Calibration curve created by plotting the fluorescence value at each cycle against the concentration of reference nucleic acid and the linear regression was calculated (FIG. 4B). Fluorescence measured over 40 PCR cycles during amplification of a 92 bp fragment from serial dilutions of a stock lambda DNA using a FAM-tagged hydrolysis probe (FIG. 4C).

FIG. 5: Fluorescence measured over 40 PCR cycles during amplification of a 92 bp fragment from serial dilutions of a stock lambda DNA using a FAM-tagged hydrolysis probe (FIG. 5A) or the intercalating dye Eva green (FIG. 5B).

FIG. 6: Fluorescence measured over 40 PCR cycles during amplification of a 92 bp fragment (FIG. 6A), a 501 bp fragment (FIG. 6B) or a 1000 bp fragment (FIG. 6C) from serial dilutions of a stock lambda. DNA using a FAM-tagged hydrolysis probe.

FIG. 7: Fluorescence measured over 40-45 PCR cycles during amplification of a 90 bp fragment with 75% CG composition (FIG. 7A) or 75% AT composition (FIG. 7B) from serial dilutions of a stock synthetic oligonucleotide using a FAM-tagged hydrolysis probe.

FIG. 8: Fluorescence measured over 45 PCR cycles for serial dilutions of reference nucleic acids tagged with FAM (FIG. 8A), HEX (FIG. 8B). Texas Red (FIG. 8C) and TYE665 (FIG. 8D). Calibration curves created for FAM (FIG. 8E), HEX (FIG. 8F), Texas Red (FIG. 8G) and TYE665 (FIG. 8H) by plotting the fluorescence value at each cycle against the concentration of fluorophore-tagged reference nucleic acid.

FIG. 9: Fluorescence measured over 45 PCR cycles during amplification from dilutions of lambda DNA using FAM-tagged hydrolysis probe (FIG. 9A), HEX-(black) or JOE-(grey) tagged hydrolysis probes (FIG. 9B), Alexa 594-(black) or Texas Red-(grey) tagged hydrolysis probes (FIG. 9C), or Cy5-(black) or TYE665-(grey) tagged hydrolysis probes (FIG. 9D).

FIG. 10: Fluorescence measured over 40 PCR cycles during amplification of a 92 bp fragment from serial dilutions of lambda DNA using FAM-, HEX- or Texas Red-tagged hydrolysis probes individually (black) or together (grey), and detected in the green (FAM) channel (FIG. 10A), the yellow (HEX) channel (FIG. 10B) or the orange (Texas Red) channel (FIG. 10C).

FIG. 11: Fluorescence measured over 40 PCR cycles during amplification of miRNA using a FAM-tagged probe.

### DEFINITIONS

A nucleic acid in the co text of the present invention is a molecule that is composed of chains of nucleotides. As used herein the term is intended to encompass DNA, RNA and their variants and derivatives. A nucleic acid may be double or single stranded.

A target nucleic acid in the context of the present invention is a nucleic acid of interest.

Amplification of nucleic acids sequences may be conveniently accomplished by Polymerase Chain Reaction (FOR) but may also be accomplished by another suitable method such as ligase chain reaction. In the context of the present specification the terms “Polymerase Chain Reaction” and its acronym “PCR” are used according to their ordinary meaning as understood by those skilled in the art. Examples of PCR methods can be found in common molecular biology textbooks and reference manuals used in the art. For example PCR Technology: Principles and Applications for DNA Amplification (1989) Ed H A Erlich. Stockton Press, New York. In order to optimise the PCR amplification, the primers can be used at different concentrations and ratios. Selection of these and other variables would be appreciated and within the competency of persons skilled in the art.

An amplicon in the context of the present invention is a nucleic acid that is formed by amplification reactions such as those performed by PCR or ligase chain reactions. In one context, the amplicon may be the amplification product of the “target nucleic acid”.

Real time PCR in the context of the present invention is a laboratory technique based on PCR, which is used to simultaneously amplify and detect/quantify amplicons.

Multiplex PCR in the context of the present invention is a laboratory technique based on PCR consisting of multiple primer sets and probes within a single PCR mixture to produce multiple amplicons that are specific to different target nucleic acids.

A probe in the context of the present invention is a nucleic acid of variable length which is used to detect the presence of a target nucleic acid that is complementary to the sequence in the probe. Probes include, but are not limited to hydrolysis probes, molecular beacons and Scorpion probes.

A primer in the context of the present invention is a nucleic acid capable of acting as a point of initiation of nucleotide synthesis.

A universal reference nucleic acid in the context of the present invention encompasses any nucleic acid useful in the preparation of a calibration curve for quantification of target nucleic acids, wherein the same universal reference nucleic acid may be used to quantify different target nucleic acids. The universal reference nucleic acid may be entirely synthetic or may be obtained from natural sources of nucleic acid.

A fluorophore in the context of the present invention is a fluorescent chemical molecule that can re-emit light upon light excitation.

A reference gene in the context of the present invention is typically a constitutive gene that is required for the maintenance of basal cellular function. Such genes are found in all cells. Some reference genes are expressed at relatively constant levels however other reference genes may vary in expression depending on experimental conditions used.

A calibration curve in the context of the present invention is a plot of fluorescence against quantity of fluorophore-tagged universal reference nucleic acid.

A standard curve in the context of the present invention is a plot of cycle threshold (Ct or Cq) against serial dilutions of a synthetic gene of interest or input nucleic acid.

Serial dilution in the context of the present invention refers to any form of dilution necessary to prepare a calibration curve or standard curve covering a range of concentrations of a fluorophore-tagged universal reference nucleic acid or synthetic gene of interest.

In the context of the present invention “dsDNA” refers to double stranded DNA, “bp” refers to base pairs, “dNTP” refers to deoxynucleotide triphosphate, “RNA” refers to ribonucleic acid. “tRNA” refers to transfer RNA, “rRNA” refers to ribosomal RNA, “siRNA” refers to small interfering RNA, “miRNA” refers to micro RNA, “mRNA” refers to messenger RNA and “cDNA” refers to complementary DNA.

The term “AccuCal-P” in the context of the present application refers to a universal reference nucleic acid tagged with any appropriate fluorophore.

The term “AccuCal-D” in the context of the present invention refers to a universal reference nucleic acid tagged using any appropriate fluorescent intercalating dye.

The term “AccuBeacon” in the context of the present invention refers to a universal reference nucleic acid tagged with any appropriate fluorophore, wherein the nucleic acid forms a hairpin structure.

### PREFERRED EMBODIMENT OF THE INVENTION

The present invention has been motivated by the lack of accurate and efficient means for quantifying nucleic acid expression in control and treatment animal/human groups. It has also been motivated by the fact that many of the known reference genes used in gene expression studies, change expression levels in response to experimental conditions or treatments, thus skewing results.

An advantage of the present invention is that the described methods dispose of the need for reference genes or synthetic reference genes used to normalise data and quantify gene expression.

In the novel approach described herein, known quantities of a fluorophore-tagged universal reference nucleic acid are used to generate a calibration curve. The calibration curve is generated by serially diluting the fluorophore-tagged universal reference nucleic acid and plotting the fluorescent levels against the concentration of the fluorophore-tagged reference nucleic acid. No amplification of the universal reference nucleic acid is necessary to produce the calibration curve. This contrasts with current methodologies for assessing gene expression whereby the test sample and the reference gene/synthetic reference gene are both amplified either side by side or combined in one reaction mixture.

The same calibration curve, once prepared, can be used numerous times if required to quantify more than one target nucleic acid. The fluorophore-tagged universal reference nucleic acids used for the preparation of the calibration curve are stable over time, for example over a period of about one month, and repeated freezing and thawing of the solutions. This enables the preparation and storage of fluorophore-tagged universal reference nucleic acid solutions ahead of any experimental requirements.

The fluorophore-tagged universal reference nucleic acid used in the methods of the present invention is not what is described as a “reference gene” or a “synthetic reference gene”, i.e. it does not need to be amplified along with the target nucleic acid.

The fluorophore-tagged universal reference nucleic acid need not have any homology with the target nucleic acid or with any reference gene sequence. However, because of particular way in which the fluorophore-tagged universal reference nucleic acid is used in the method of the present invention (Le, to set up a calibration curve following serial dilution of the fluorophore-tagged universal reference nucleic acid), the fluorophore-tagged universal reference nucleic acid can have a degree of homology or can even be identical to a target nucleic acid sequence or a reference gene, or smaller parts thereof.

Each universal reference nucleic acid is tagged with a defined number of fluorophores, typically numbering one. Similarly, the probe is tagged with a known number of fluorophores, which allows for direct comparison between the fluorescence generated by the fluorophore-tagged universal reference nucleic acid and the fluorescence generated by the fluorophore-tagged probe-bound target nucleic acid. As such, the length and/or composition of the fluorophore-tagged universal reference nucleic acid is irrelevant, as long as a defined amount of fluorophore can be determined. In the embodiments described herein, the fluorophore-tagged reference nucleic acid is 21 bp in length and varies between 30%-80% GC content, but it will be appreciated by those skilled in the art, that any length or GC content of fluorophore-tagged reference nucleic acid will provide similar results.

If a fluorophore-tagged universal reference nucleic acid is obtained from a biological source, natural or otherwise, it may be obtained using restriction enzymes to obtain a suitable fragment. Synthetic fluorophore-tagged universal reference nucleic acids may also be conveniently used and may be simply prepared by known techniques such as for example those described in Sambrook et al. Molecular Cloning: A Laboratory Manual, 2nd ed. (1989) Cold Spring Harbor Laboratory Press, Cold Spring Harbor. N.Y. and Roe at al. DNA Isolation and Sequencing” (Essential Techniques Series) (1996) John Wiley & Sons, Inc., N.Y.

The same fluorophore-tagged universal reference nucleic acid can be used for numerous nucleic acid targets. Alternatively multiple calibration curves can be generated using differing fluorophore-tagged universal reference nucleic acid and similarly differing fluorophore-tagged probes to quantify nucleic acids in multiplex PCR reactions.

Thus, the method of the present invention is advantageous as it disposes of the need to amplify a reference gene and constantly run assays for reference genes or synthetic reference genes to normalize nucleic acid expression data within each experiment. One calibration curve can be prepared and used to quantify more than one target nucleic acid, which can vary in size and sequence, thereby cutting the cost and time when compared to conventional gene expression assays.

As a general guide for preparing a calibration curve to be used in the method of the present invention, the fluorophore-tagged universal reference nucleic acid is serially diluted at a range of concentrations in duplicate using the same reaction buffer as that used for the target nucleic acid and the same reaction tubes and subjected to the same amplification conditions as the target nucleic acid.

The present invention will now be described in more detail with reference to specific but non-limiting examples describing specific compositions and methods of use. It is to be understood, however, that the detailed description of specific procedures, compositions and methods is included solely for the purpose of exemplifying the present invention. It should not be understood in any way as a restriction on the broad description of the inventive concept as set out above.

### EXAMPLES

### Example 1

### Use of AccuCal-P to Quantitate a Range of Amplified Lambda DNA

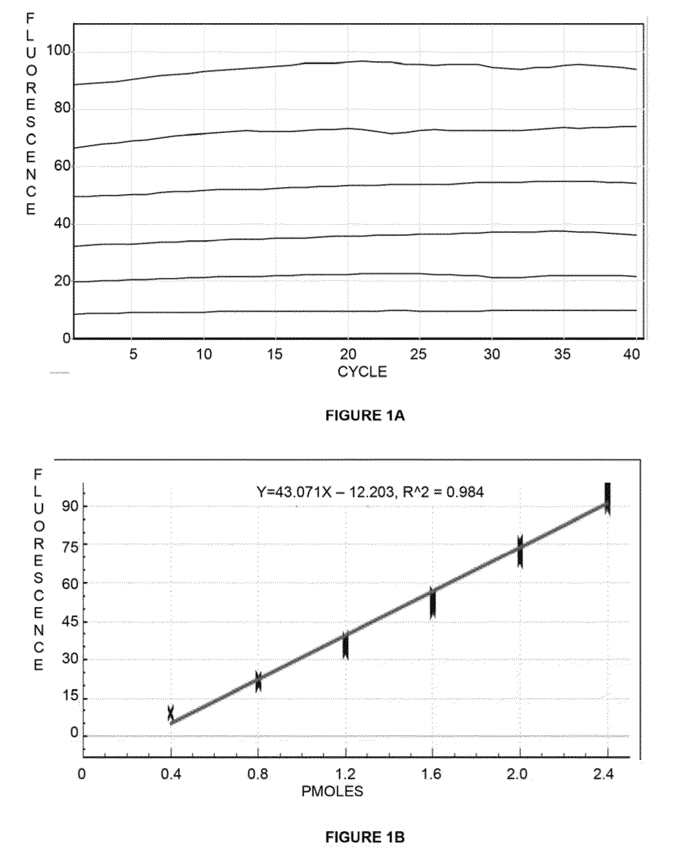

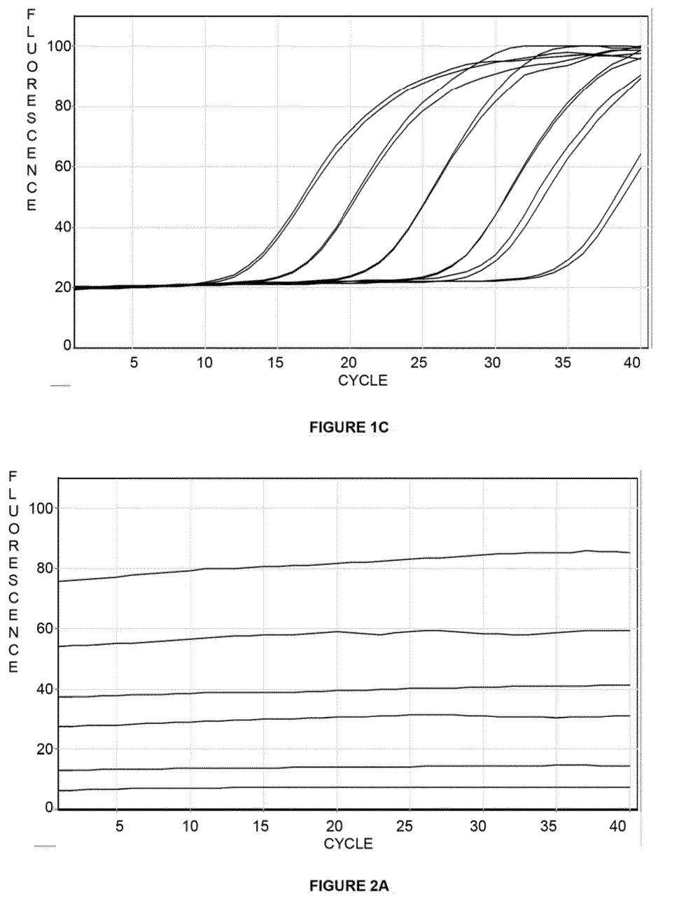

A FAM-tagged reference nucleic acid (21 bp and 30% GC—AccuCal-P) was diluted to 0 nM, 20 nM, 40 nM, 60 nM, 80 nM, 100 nM and 120 nM and fluorescence was measured over 40 PCR cycles (FIG. 1A). A calibration curve was created by plotting the fluorescence value at each cycle against the concentration of reference nucleic acid and the linear regression was calculated (FIG. 1B). A range of ten-fold serial dilutions of a stock lambda DNA was amplified over 40 PCR cycles, with a 92 bp fragment of amplified lambda DNA detected using a FAM-tagged hydrolysis probe (FIG. 1C). Each PCR was performed in triplicate. The theoretical amount of seeded nucleic acid ranged from 4.5×10⁷ to 4.5×10¹ copies/PCR, along with a negative control (NTC). The initial amount of nucleic acid seeded into each of the amplification reactions was quantitated using the calibration curve and the individual efficiency of each reaction over the exponential portion of the amplification curve using a published algorithm (Tichopad at al. 2003, Nucl. Acids Res., 31(20): e122). From the calibration curve, the pmols of DNA in each amplification reaction was determined over the exponential portion of the amplification curve and the mean initial input DNA was calculated using the equation pmz=pm/Eⁿ, where pmz is pmols at time zero, E is efficiency and n is cycle number. The pmz is converted into copies/PCR using pmz×6.022²³×10⁻¹².

The mean amount of starting nucleic acid, and standard error of the mean, was determined for each triplicate (Table 1).

TABLE 1

| Theoretical amount | Mean calculated | Standard error |
|---|---|---|
| seeded (c/PCR) | amount (c/PCR) | of mean |
| 4.5 × 10¹ | 4.59 × 10¹ | 1.22 × 10¹ |
| 4.5 × 10² | 1.91 × 10² | 3.95 × 10¹ |
| 4.5 × 10³ | 3.87 × 10³ | 1.11 × 10³ |
| 4.5 × 10⁴ | 1.86 × 10⁴ | 4.51 × 10³ |
| 4.5 × 10⁵ | 6.17 × 10⁵ | 1.56 × 10⁵ |
| 4.5 × 10⁶ | 1.01 × 10⁷ | 2.67 × 10⁶ |
| 4.5 × 10⁷ | 7.89 × 10⁷ | 1.88 × 10⁷ |
| NTC | 0 | 0 |

### Example 2

### Use of AccuCal-P to Quantitate a Range of Amplified Lambda DNA

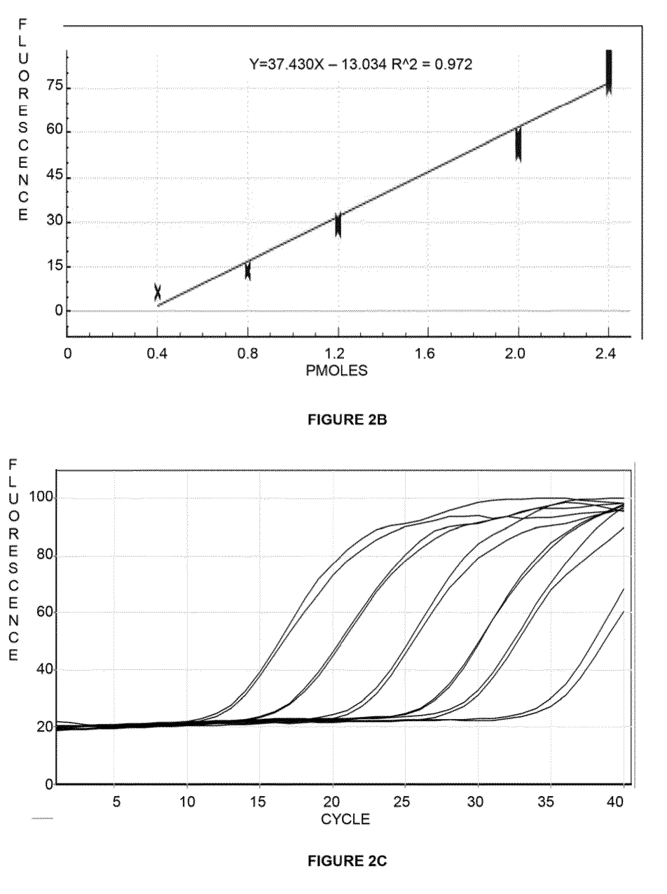

A FAM-tagged reference nucleic acid (21 bp and 40% GC—AccuCal-PI was diluted to 0 nM, 20 nM, 40 nM, 60 nM, 80 nM, 100 nM and 120 nM and fluorescence was measured over 40 PCR cycles (FIG. 2A). A calibration curve was created by plotting the fluorescence value at each cycle against the concentration of reference nucleic acid and the linear regression was calculated (FIG. 2B). A range of ten-fold serial dilutions of a stock lambda DNA was amplified over 40 PCR cycles, with a 92 bp fragment of amplified lambda DNA detected using a FAM-tagged hydrolysis probe (FIG. 2C). Each PCR was performed in triplicate. The theoretical amount of seeded nucleic acid ranged from 4.5×10⁷ to 4.5×10¹ copies/PCR, along with a negative control (NTC). The initial amount of nucleic acid seeded into each of the amplification reactions was quantitated using the calibration curve and the individual efficiency of each reaction over the exponential portion of the amplification curve using a published algorithm (Tichopad at al. 2003, Nucl. Acids Res., 31(20): e122). From the calibration curve, the pmols of DNA in each amplification reaction was determined over the exponential portion of the amplification curve and the mean initial input DNA was calculated using the equation pmz=pm/Eⁿ, where pmz is pmols at time zero, E is efficiency and n is cycle number. The pmz is converted into copies/PCR using pmz×6.022²³×10⁻¹².

The mean amount of starting nucleic acid, and standard error of the mean, was determined for each triplicate (Table 2).

TABLE 2

| Theoretical amount | Mean calculated | Standard error |
|---|---|---|
| seeded (c/PCR) | amount (c/PCR) | of mean |
| 4.5 × 10¹ | 6.51 × 10¹ | 1.23 × 10¹ |
| 4.5 × 10² | 2.95 × 10² | 7.79 × 10¹ |
| 4.5 × 10³ | 9.07 × 10³ | 2.50 × 10³ |
| 4.5 × 10⁴ | 1.89 × 10⁴ | 4.50 × 10³ |
| 4.5 × 10⁵ | 8.66 × 10⁵ | 2.76 × 10⁵ |
| 4.5 × 10⁶ | 1.43 × 10⁷ | 3.71 × 10⁶ |
| 4.5 × 10⁷ | 9.68 × 10⁷ | 2.34 × 10⁷ |
| NTC | 0 | 0 |

### Example 3

### Use of AccuCal-P to Quantitate a Range of Amplified Lambda DNA

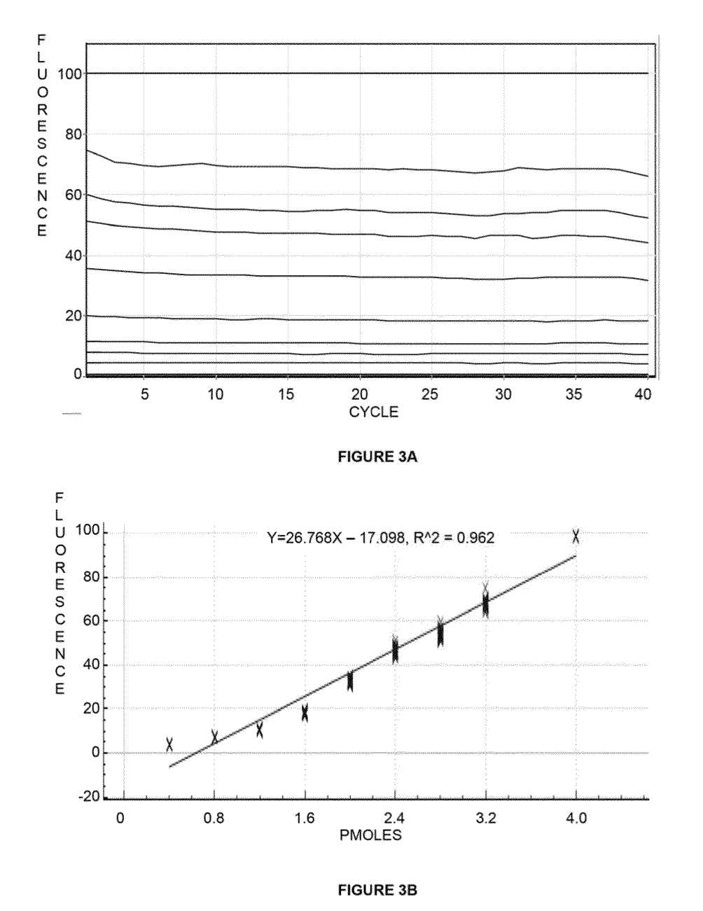

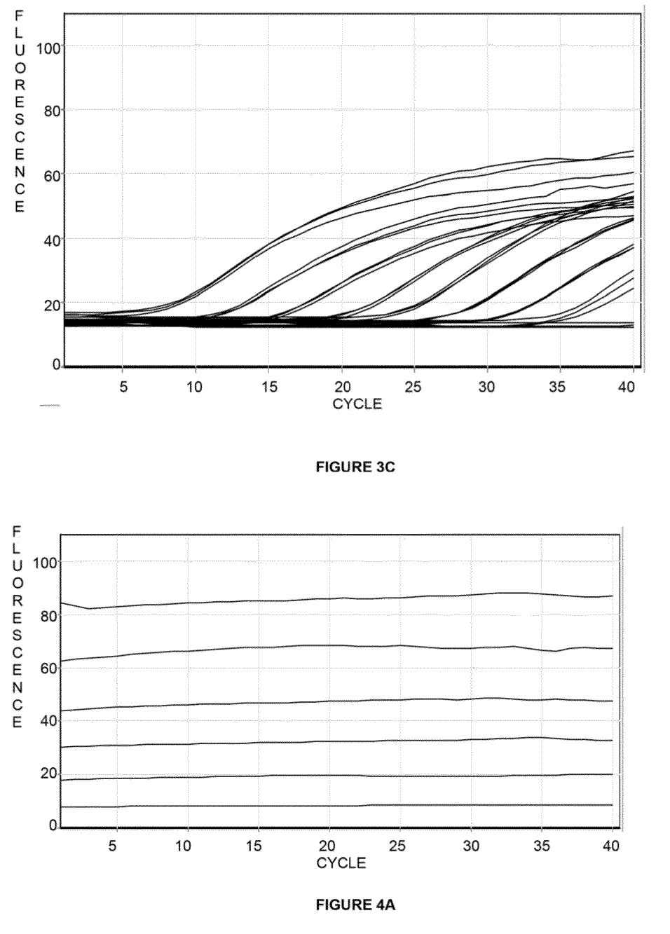

A FAM-tagged reference nucleic acid (21 bp and 62% GC—AccuCal-P) was diluted to 0 nM, 20 nM, 40 nM, 60 nM, 80 nM, 100 nM, 120 nM, 140 nM, 160 nM and 200 nM, and fluorescence was measured over 40 PCR cycles (FIG. 3A). A calibration curve was created by plotting the fluorescence value at each cycle against the concentration of reference nucleic acid and the linear regression was calculated (FIG. 3B). A range of ten-fold serial dilutions of a stock lambda DNA was amplified over 40 PCR cycles, with a 92 bp fragment of amplified lambda DNA detected using a FAM-tagged hydrolysis probe (FIG. 3C). Each PCR was performed in triplicate. The theoretical amount of seeded nucleic acid ranged from 4.5×10⁷ to 4.5×10¹ copies/PCR, along with a negative control (NTC). The initial amount of nucleic acid seeded into each of the amplification reactions was quantitated using the calibration curve and the individual efficiency of each reaction over the exponential portion of the amplification curve using a published algorithm (Tichopad et al. 2003, Nucl. Adds Res., 31(20): e122). From the calibration curve, the pmols of DNA in each amplification reaction was determined over the exponential portion of the amplification curve and the mean initial input DNA was calculated using the equation pmz=pm/Eⁿ, where pmz is pmols at time zero, E is efficiency and n is cycle number. The pmz is converted into copies/PCR using pmz×6.022²³×10⁻¹².

The mean amount of starting nucleic acid, and standard error of the mean, was determined for each triplicate (Table 3).

TABLE 3

| Theoretical amount | Mean calculated | Standard error |
|---|---|---|
| seeded (c/PCR) | amount (c/PCR) | of mean |
| 4.5 × 10¹ | 8.58 × 10¹ | 3.06 × 10¹ |
| 4.5 × 10² | 6.22 × 10² | 1.15 × 10² |
| 4.5 × 10³ | 7.48 × 10³ | 1.48 × 10³ |
| 4.5 × 10⁴ | 6.42 × 10⁴ | 1.26 × 10⁴ |
| 4.5 × 10⁵ | 4.44 × 10⁵ | 8.09 × 10⁴ |
| 4.5 × 10⁶ | 1.30 × 10⁷ | 2.42 × 10⁶ |
| 4.5 × 10⁷ | 9.19 × 10⁷ | 1.68 × 10⁷ |
| NTC | 0 | 0 |

### Example 4

### Use of AccuCal-P to Quantitate a Range of Amplified Lambda DNA

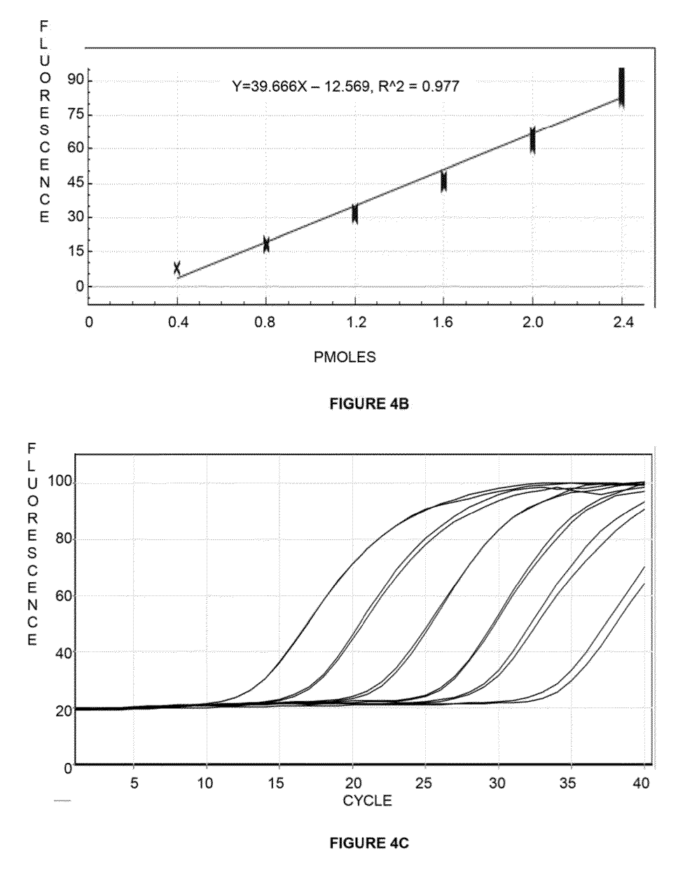

A FAM-tagged reference nucleic acid (21 bp and 80% GC—AccuCal-P) was diluted to 0 nM, 20 nM, 40 nM, 60 nM, 80 nM, 100 nM and 120 nM and fluorescence was measured over 40 PCR cycles (FIG. 4A). A calibration curve was created by plotting the fluorescence value at each cycle against the concentration of reference nucleic acid and the linear regression was calculated (FIG. 4B). A range of ten-fold serial dilutions of a stock lambda DNA was amplified over 40 PCR cycles, with a 92 bp fragment of amplified lambda DNA detected using a FAM-tagged hydrolysis probe (FIG. 4C). Each PCR was performed in triplicate. The theoretical amount of seeded nucleic add ranged from 4.5×10⁷ to 4.6×10¹ copies/PCR, along with a negative control (NTC). The initial amount of nucleic acid seeded into each of the amplification reactions was quantitated using the calibration curve and the individual efficiency of each reaction over the exponential portion of the amplification curve using a published algorithm (Tichopad et al. 2003, Nucl. Acids Res., 31(20): e122). From the calibration curve, the pmols of DNA in each amplification reaction was determined over the exponential portion of the amplification curve and the mean initial input DNA was calculated using the equation pmz=pm/Eⁿ, where pmz is pmols at time zero. E is efficiency and n is cycle number. The pmz is converted into copies/PCR using pmz×6.022²³×10⁻¹².

The mean amount of starting nucleic acid, and standard error of the mean, was determined for each triplicate (Table 4).

TABLE 4

| Theoretical amount | Mean calculated | Standard error |
|---|---|---|
| seeded (c/PCR) | amount (c/PCR) | of mean |
| 4.5 × 10¹ | 1.26 × 10² | 3.63 × 10¹ |
| 4.5 × 10² | 3.71 × 10² | 8.98 × 10¹ |
| 4.5 × 10³ | 7.67 × 10³ | 1.90 × 10³ |
| 4.5 × 10⁴ | 3.38 × 10⁴ | 7.96 × 10³ |
| 4.5 × 10⁵ | 5.76 × 10⁵ | 1.53 × 10⁵ |
| 4.5 × 10⁶ | 1.33 × 10⁷ | 3.27 × 10⁶ |
| 4.5 × 10⁷ | 8.30 × 10⁷ | 1.93 × 10⁷ |
| NTC | 0 | 0 |

### Example 5

### Use of AccuCal-D, AccuCal-P and AccuBeacon to Quantitate a Range of Amplified Lambda DNA

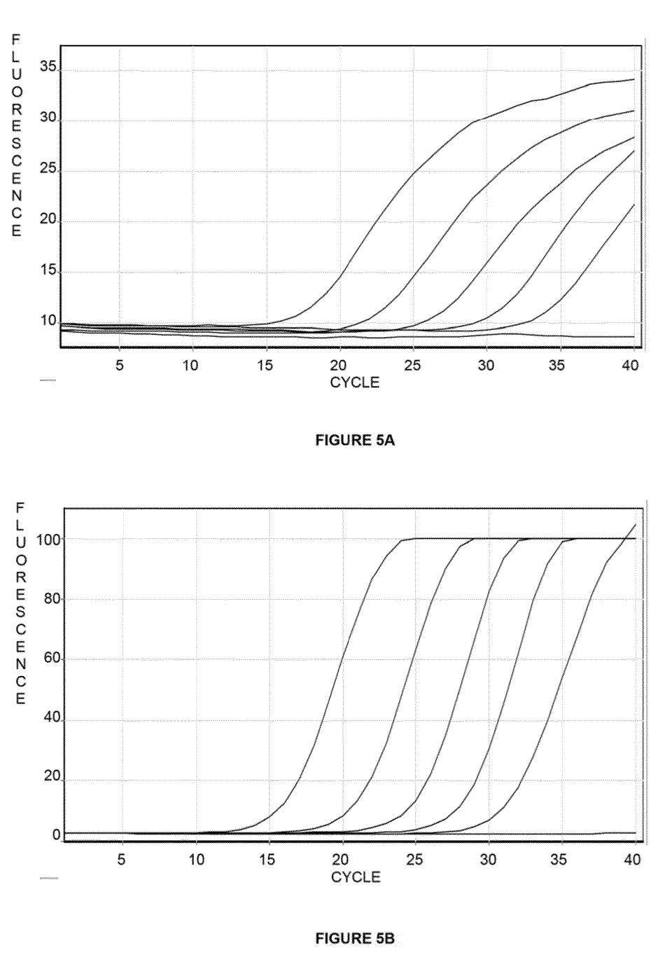

Three reference nucleic acids were used to independently quantitate a range of lambda DNA: AccuCal-D (21 bp and 62% GC reference nucleic acid tagged with Eva green), AccuCal-P (21 bp and 62% GC reference nucleic acid tagged with FAM) and AccuBeacon (21 bp and 62% Sc hairpin reference nucleic acid tagged with FAM). Each reference nucleic acid (AccuCal-D, AccuCal-P and AccuBeacon) was diluted to 0 nM, 20 nM, 40 nM, 60 nM, 80 nM, 100 nM, 140 nM and 200 nM. A 92 bp fragment was amplified from ten-fold serial dilutions of lambda DNA, ranging from 4.5×10⁶ to 4.5×10² and an NTC, and detected using a FAM-tagged hydrolysis probe (FIG. 5A) or the intercalating dye Eva green (FIG. 5B). The amount of starting nucleic acid input was calculated using AccuCal-D, AccuCal-P or AccuBeacon as a calibrator and compared to the theoretical amount of nucleic acid seeded into each PCR (Tables 5A and 5A).

TABLE 5A

| Theoretical | Mean calculated amount (c/PCR) in PCRs | | |
|---|---|---|---|
| amount seeded | detected with Eva-green intercalating dye | | |
| (c/PCR) | AccuCal-D | AccuCal-P | AccuBeacon |
| 4.5 × 10⁶ | 6.14 × 10⁶ | 3.67 × 10⁶ | 9.64 × 10⁶ |
| 4.5 × 10⁵ | 4.05 × 10⁵ | 2.44 × 10⁵ | 6.74 × 10⁵ |
| 4.5 × 10⁴ | 8.01 × 10⁴ | 4.77 × 10⁴ | 1.22 × 10⁵ |
| 4.5 × 10³ | 5.38 × 10³ | 3.17 × 10³ | 7.36 × 10³ |
| 4.5 × 10² | 3.19 × 10² | 1.90 × 10² | 4.86 × 10² |
| NTC | 0 | 0 | 0 |

TABLE 5A

| Theoretical | Mean calculated amount (c/PCR) in PCRs | | |
|---|---|---|---|
| amount seeded | detected with FAM hydrolysis probe | | |
| (c/PCR) | AccuCal-D | AccuCal-P | AccuBeacon |
| 4.5 × 10⁶ | 7.42 × 10⁶ | 4.36 × 10⁶ | 9.94 × 10⁶ |
| 4.5 × 10⁵ | 4.40 × 10⁵ | 2.57 × 10⁵ | 5.53 × 10⁵ |
| 4.5 × 10⁴ | 5.45 × 10⁴ | 3.19 × 10⁴ | 6.97 × 10⁴ |
| 4.5 × 10³ | 6.61 × 10³ | 3.87 × 10³ | 8.47 × 10³ |
| 4.5 × 10² | 1.67 × 10³ | 9.87 × 10² | 2.31 × 10³ |
| NTC | 0 | 0 | 0 |

### Example 6

### Use of AccuCal-D or AccuCal-P to Quantify a Range of Amplicon Sizes

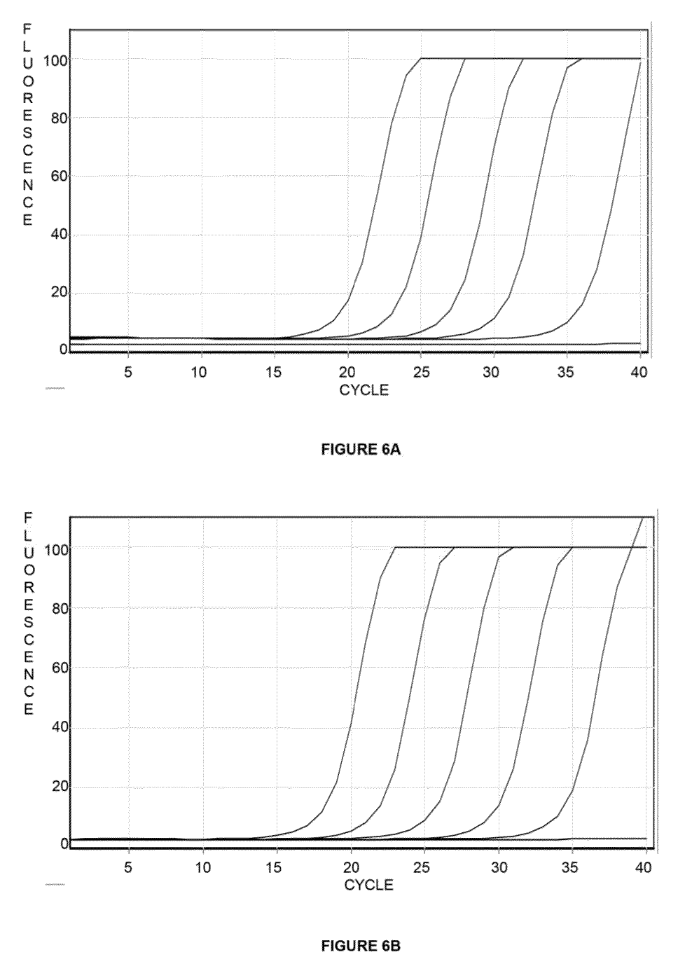

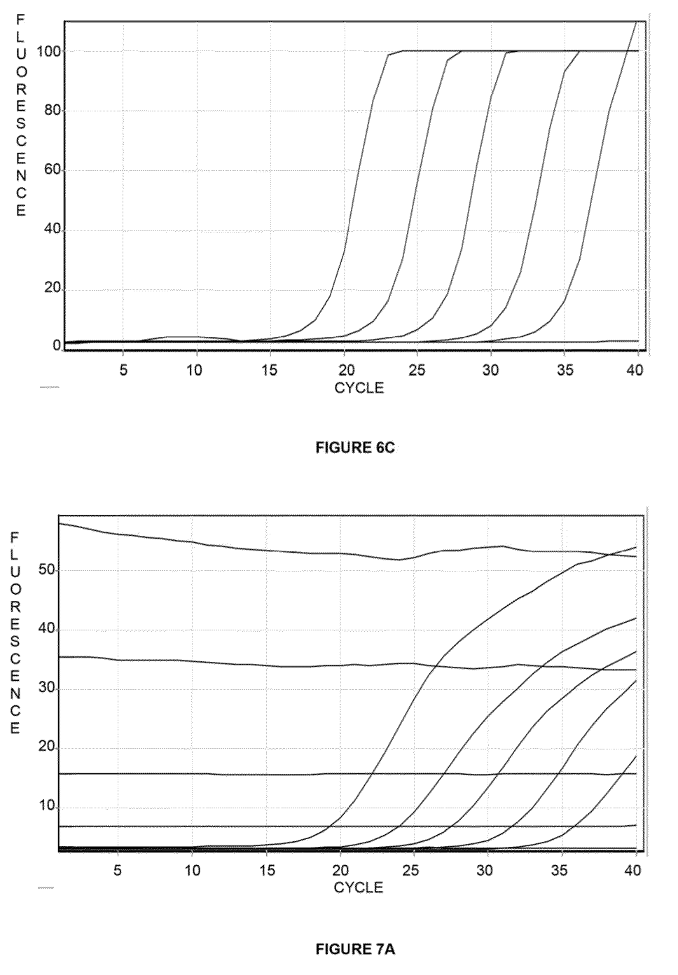

Primers were designed to amplify 92 bp, 501 bp and 1000 bp amplicons from lambda DNA (FIGS. 6A, 6B and 6C, respectively). Each fragment was amplified from ten-fold serial dilutions of lambda DNA, ranging from 4.5×10⁵ to 4.5×10¹ and an NTC. The amount of starting nucleic acid input was calculated using either AccuCal-P (21 bp and 62% GC reference nucleic acid tagged with FAM) or AccuCal-D (21 bp and 62% GC reference nucleic acid tagged with EvaGreen) as a calibrator (Table 6).

TABLE 6

| Theoretical | | | | | | |
|---|---|---|---|---|---|---|
| amount | Mean calculated amount (c/PCR) | | | | | |
| seeded | 92 bp | | 501 bp | | 1000 bp | |
| (c/PCR) | AccuCal-P | AccuCal-D | AccuCal-P | AccuCal-D | AccuCal-P | AccuCal-D |
| 4.5 × 10⁵ | 1.8 × 10⁵ | 1.89 × 10⁵ | 6.05 × 10⁵ | 6.40 × 10⁵ | 9.42 × 10⁵ | 1.17 × 10⁸ |
| 4.5 × 10⁴ | 2.17 × 10⁴ | 2.31 × 10⁴ | 4.85 × 10⁴ | 5.20 × 10⁴ | 4.14 × 10⁴ | 1.09 × 10⁴ |
| 4.5 × 10³ | 1.21 × 10³ | 1.29 × 10³ | 4.21 × 10³ | 4.49 × 10³ | 8.83 × 10³ | 4.32 × 10³ |
| 4.5 × 10² | 1.31 × 10² | 1.40 × 10² | 2.52 × 10² | 2.69 × 10² | 8.61 × 10² | 3.11 × 10² |
| 4.5 × 10¹ | 8.47 × 10⁰ | 8.95 × 10⁰ | 1.44 × 10¹ | 1.53 × 10¹ | 3.49 × 10¹ | 3.70 × 10¹ |
| NTC | 0 | 0 | 0 | 0 | 0 | 0 |

### Example 7

### Use of AccuCal-P to Quantitate Nucleic Acids of Varying Composition

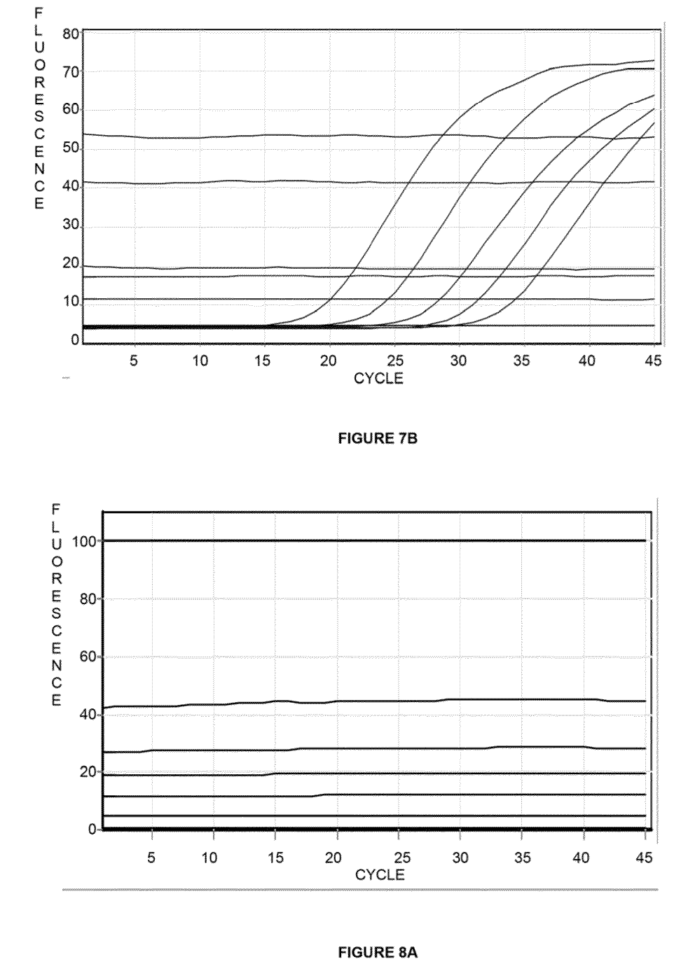

Synthetic target nucleic acids were designed from which a 90 bp amplicon could be amplified that was either 75% CG composition (FIG. 7A) or 75% AT composition (FIG. 7B). The synthetic templates were diluted ten-fold so that a known amount of template, ranging from 1×10⁷ to 1×10³ plus NTC, was seeded per PCR, AccuCAL-P (21 bp and 62% GC reference nucleic acid tagged with FAM) was diluted to 0 nM, 20 nM, 40 nM, 80 nM, 100 nM and 120 nM. The amount of starting nucleic acid input was calculated using AccuCAL-P as a calibrator for both GC rich template (Table 7A) and AT rich template (Table 7B).

TABLE 7A

| Theoretical amount | Mean calculated | Standard error |
|---|---|---|
| seeded (c/PCR) | amount (c/PCR) | of mean |
| 4.5 × 10³ | 1.34 × 10⁴ | 3.93 × 10³ |
| 4.5 × 10⁴ | 3.14 × 10⁴ | 9.94 × 10³ |
| 4.5 × 10⁵ | 3.03 × 10⁵ | 9.98 × 10⁴ |
| 4.5 × 10⁶ | 1.83 × 10⁶ | 5.71 × 10⁵ |
| 4.5 × 10⁷ | 1.51 × 10⁷ | 4.72 × 10⁶ |
| NTC | 0 | 0 |

TABLE 7B

| Theoretical amount | Mean calculated | Standard error |
|---|---|---|
| seeded (c/PCR) | amount (c/PCR) | of mean |
| 4.5 × 10³ | 1.98 × 10³ | 6.52 × 10² |
| 4.5 × 10⁴ | 1.89 × 10⁴ | 6.58 × 10³ |
| 4.5 × 10⁵ | 1.82 × 10⁵ | 6.35 × 10⁴ |
| 4.5 × 10⁶ | 7.00 × 10⁵ | 2.46 × 10⁵ |
| 4.5 × 10⁷ | 1.24 × 10⁷ | 4.40 × 10⁶ |
| NTC | 0 | 0 |

### Example 8

### Fluorescence of Various Amounts of Four Reference Nucleic Acids, Each Tagged with a Different Fluorophore, May be Detected Simultaneously in the Same Well

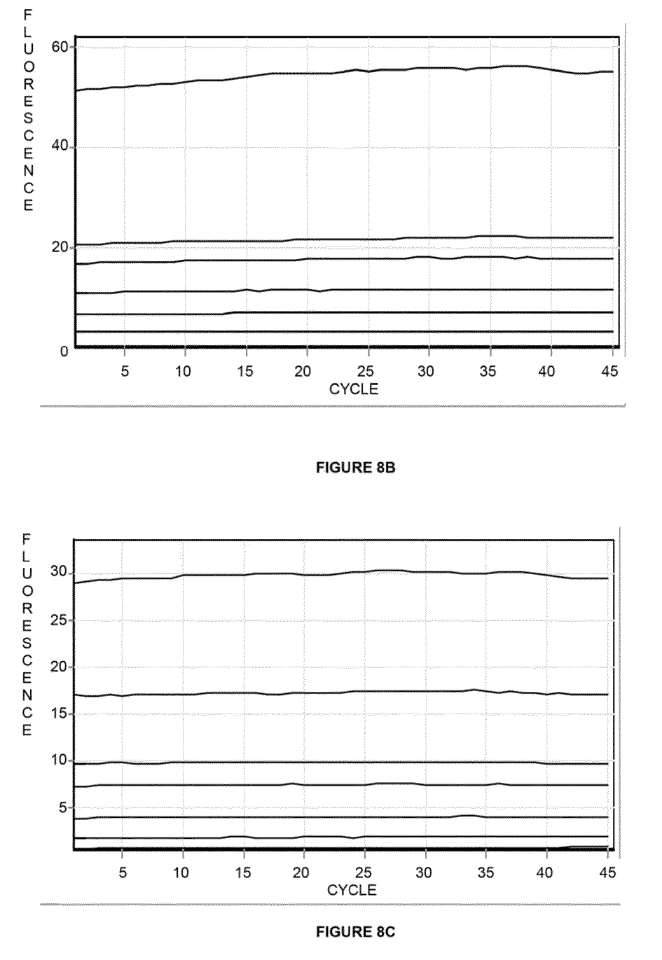

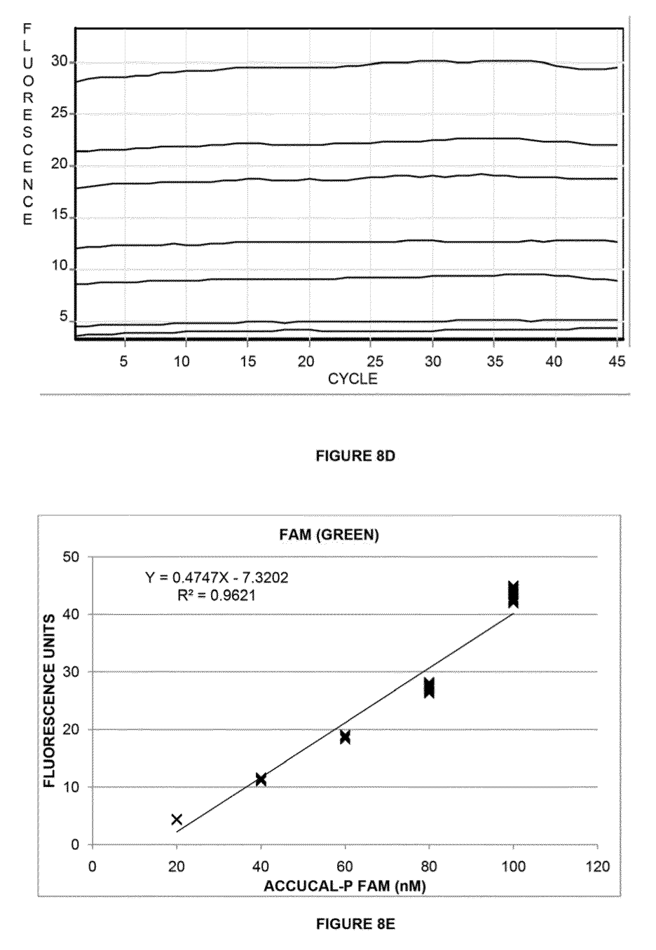

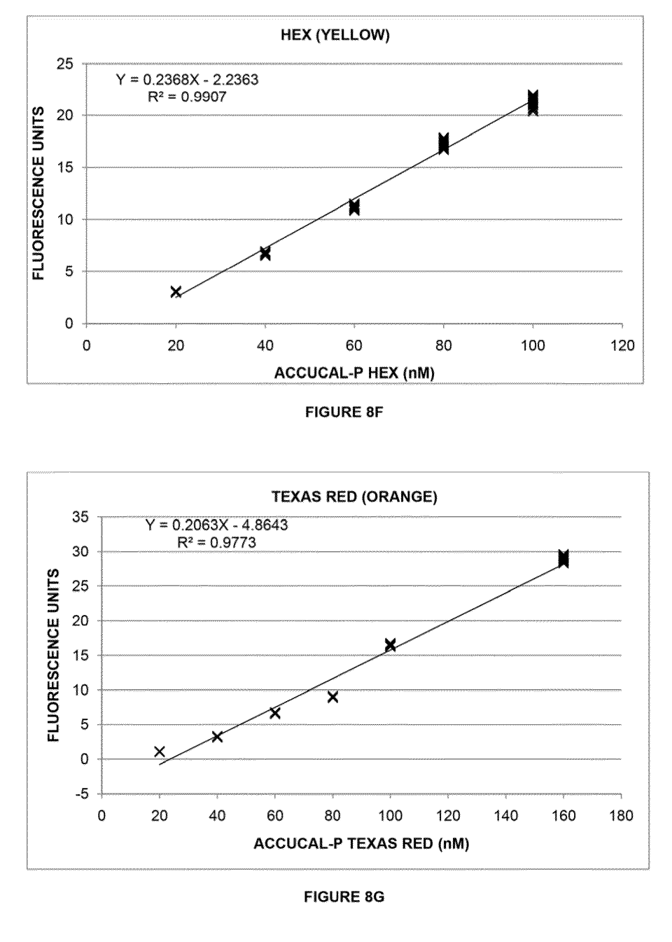

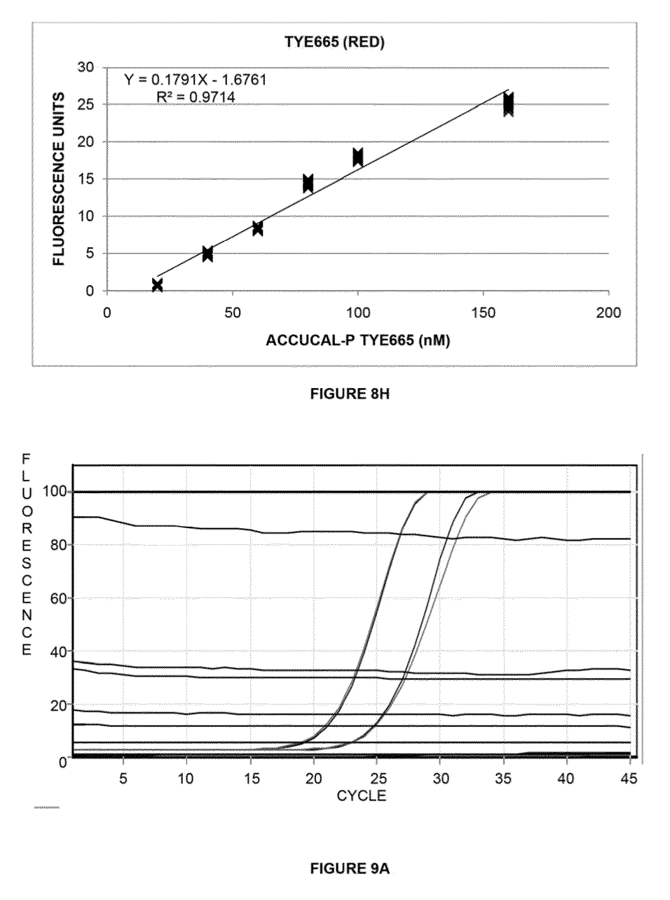

Reference nucleic acids (21 bp and 62% GC) were tagged with FAM (FIG. 8A), HEX (FIG. 8B), Texas Red (FIG. 8C) and TYE665 (FIG. 8D) and are detected in the green, yellow, orange and red filter channels, respectively. In each case, the fluorophore-tagged reference nucleic acids were diluted to 0 nM, 20 nM, 40 nM, 60 nM, 80 nM, 100 nM and 160 nM and the fluorescence was measured at the end of each annealing step of the PCR amplification cycle.

Calibration curves were generated for each of the four reference nucleic acids (FIG. 8E to FIG. 8H). The fluorescence value for every cycle for each concentration of the calibrator is plotted and the linear regression calculated. In each case, the equation and R² value for the linear regression line is displayed. The 0 nM calibrator represents the background level of fluorescence of the reagents, plastic etc and is removed from all values to provide genuine fluorescence values for each concentration of calibrator.

Three reference nucleic acids (21 bp and 62% GC), diluted at 0 nM, 20 nM, 40 nM, 60 nM, 80 nM, 100 nM, 120 nM, 140 nM, 160 nM and 200 nM, and tagged with either FAM, HEX or Texas Red were used to quantitate a range of lambda PCRs (Table 8). The FAM-tagged reference nucleic acid was used to quantitate lambda PCRs detected with a FAM-tagged hydrolysis probe. The HEX-tagged reference nucleic acid was used to quantitate lambda PCRs detected with either a HEX-tagged hydrolysis probe or a JOE-tagged hydrolysis probe. The Texas Red-tagged reference nucleic acid was used to quantitate lambda PCRs detected with either a Texas Red-tagged hydrolysis probe or an Alexa 594-tagged hydrolysis probe.

TABLE 8

| Theoretical | | | | | |
|---|---|---|---|---|---|
| amount | | | | | |
| seeded | Mean calculated amount (c/PCR) | | | | |
| (c/PCR) | FAM | JOE | HEX | Alexa 594 | Texas Red |
| 4.5 × 10⁶ | 2.72 × 10⁶ | 5.39 × 10⁶ | 2.57 × 10⁶ | 6.07 × 10⁶ | 1.20 × 10⁷ |
| 4.5 × 10⁵ | 4.44 × 10⁵ | 3.14 × 10⁵ | 1.91 × 10⁵ | 2.03 × 10⁵ | 6.57 × 10⁵ |
| 4.5 × 10⁴ | 6.42 × 10⁴ | 3.99 × 10⁴ | 2.95 × 10⁴ | 1.41 × 10⁴ | 1.32 × 10⁴ |
| 4.5 × 10³ | 7.48 × 10³ | 6.66 × 10³ | 1.86 × 10³ | 2.19 × 10³ | 1.57 × 10³ |
| 4.5 × 10² | 6.22 × 10² | 1.27 × 10³ | 6.09 × 10² | 9.83 × 10² | 4.83 × 10² |
| NTC | 0 | 0 | 0 | 0 | 0 |

### Example 9

### Use of a Reference Nucleic Acid Tagged with One Fluorophore for Quantitating Amplifications Detected Using a Different Fluorophore

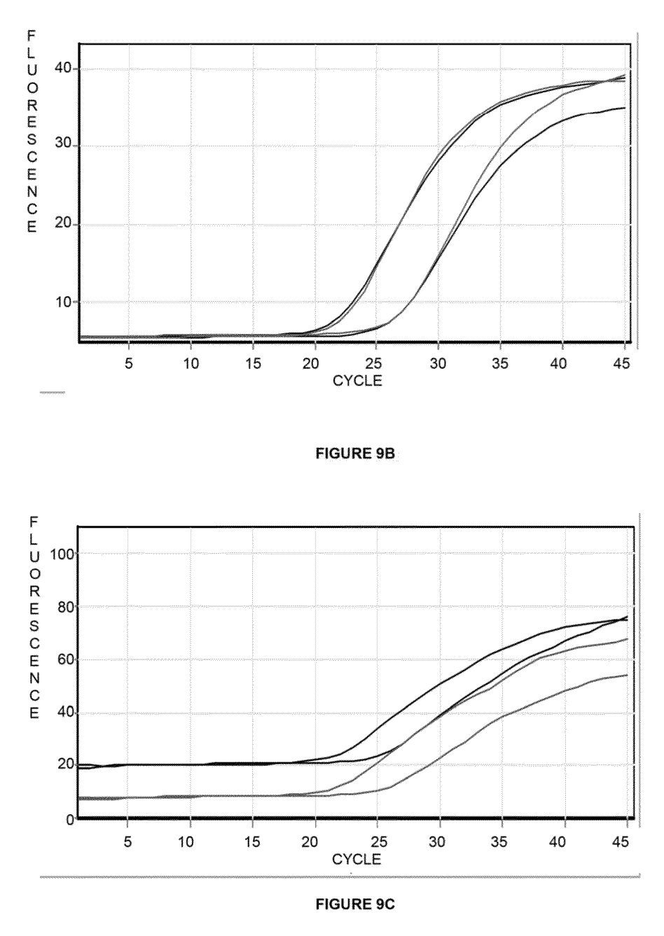

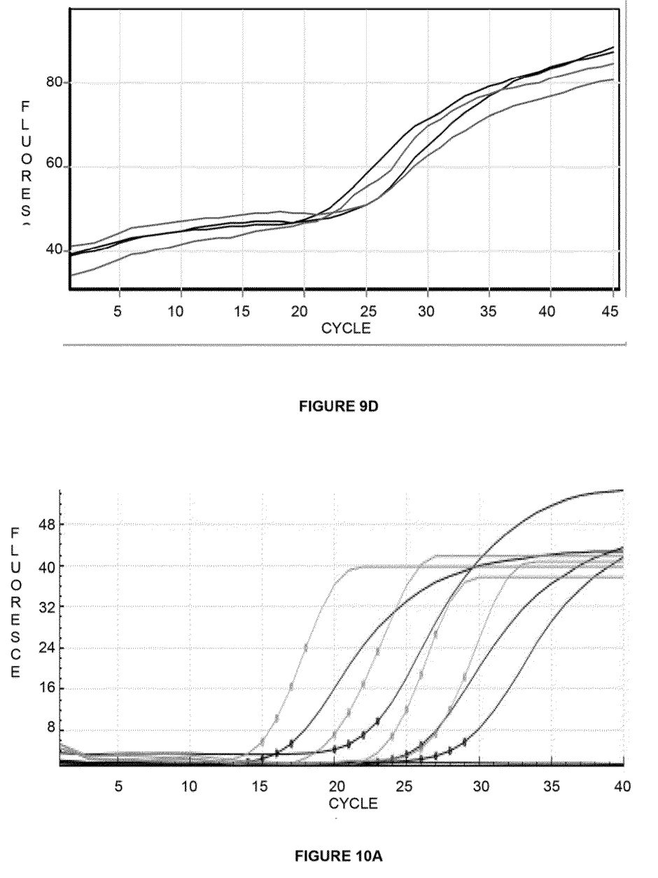

Lambda DNA (4.5×10⁵ and 4.5×10⁴ c/PCR) was amplified and detected using FAM-tagged hydrolysis probe (FIG. 9A), HEX-(black) or JOE-(grey) tagged hydrolysis probes (FIG. 9B), Alexa 594-(black) or Texas Red-(grey) tagged hydrolysis probes (FIG. 9C), or Cy5-(black) or TYE665-(grey) tagged hydrolysis probes (FIG. 9D). A FAM-tagged reference nucleic acid (21 bp and 62% GC) was diluted to 0 nM. 20 nM, 40 nM, 60 nM, 80 nM, 100 nM. 120 nM and 140 nM and used to quantitate all the amplifications (Table 9).

TABLE 9

| Theoretical | | | | | | | |
|---|---|---|---|---|---|---|---|
| amount | Mean calculated amount (c/PCR) | | | | | | |
| seeded | | | | Alexa | Texas | | |
| (c/PCR) | FAM | JOE | HEX | 594 | Red | Cy5 | TYE665 |
| 4.5 × 10⁴ | 2.61 × 10⁴ | 5.01 × 10⁴ | 5.78 × 10⁴ | 2.83 × 10⁴ | 1.82 × 10⁴ | 4.51 × 10⁴ | 1.96 × 10⁴ |
| 4.5 × 10⁵ | 4.36 × 10⁵ | 3.34 × 10⁵ | 2.25 × 10⁵ | 2.13 × 10⁵ | 2.05 × 10⁵ | 2.82 × 10⁵ | 3.32 × 10⁵ |

### Example 10

### Both AccuCal-P and Samples can be Multiplexed

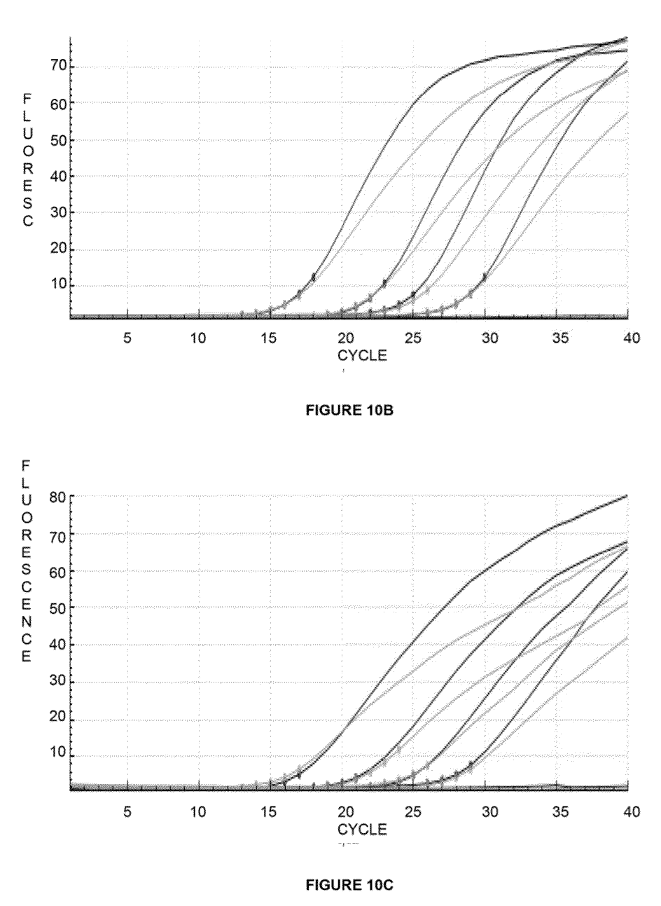

Three reference nucleic acids (21 bp and 62% GC) tagged with either FAM, HEX or Texas Red (AccuCal-P) were each diluted to 0 nM, 20 nM, 40 nM, 60 nM, 80 nM, 100 nM, 120 nM, 140 nM, 160 nM and 200 nM and combined together in one well of a PCR plate. In other wells, lambda DNA in ten-fold dilutions, ranging from 4.5×10⁶ to 4.5×10³ c/PCR plus a NTC, were amplified. Different amplicons of lambda, each approximately 100 bp in length, were amplified and detected either individually using a FAM, HEX or Texas Red tagged probe (curves shown in black), or all together in the same well (curves shown in grey) and detected in the green (FAM) channel (FIG. 10A), the yellow (HEX) channel (FIG. 10B) or the orange (Texas Red) channel (FIG. 10C). The amount of input DNA was calculated using the AccuCal-P reference nucleic acid with the same fluorophore (Table 10).

TABLE 10

| Theoretical | Mean calculated amount (c/PCR) | | | | | |
|---|---|---|---|---|---|---|
| amount seeded | FAM | | HEX | | Texas Red | |
| (c/PCR) | Alone | Multiplex | Alone | Multiplex | Alone | Multiplex |
| 4.5 × 10⁶ | 1.25 × 10⁷ | 4.44 × 10⁶ | 7.95 × 10⁶ | 7.03 × 10⁶ | 6.33 × 10⁶ | 6.03 × 10⁶ |
| 4.5 × 10⁵ | 3.65 × 10⁵ | 2.66 × 10⁵ | 1.83 × 10⁵ | 5.24 × 10⁵ | 3.41 × 10⁵ | 4.20 × 10⁵ |
| 4.5 × 10⁴ | 2.08 × 10⁴ | 2.42 × 10⁴ | 2.73 × 10⁴ | 2.55 × 10⁴ | 3.26 × 10⁴ | 3.74 × 10⁴ |
| 4.5 × 10³ | 1.36 × 10³ | 5.73 × 10³ | 2.18 × 10³ | 4.48 × 10³ | 4.00 × 10³ | 6.06 × 10³ |
| NTC | 0 | 0 | 0 | 0 | 0 | 0 |

### Example 11

### AccuCal-P can be Used to Quantitate miRNA

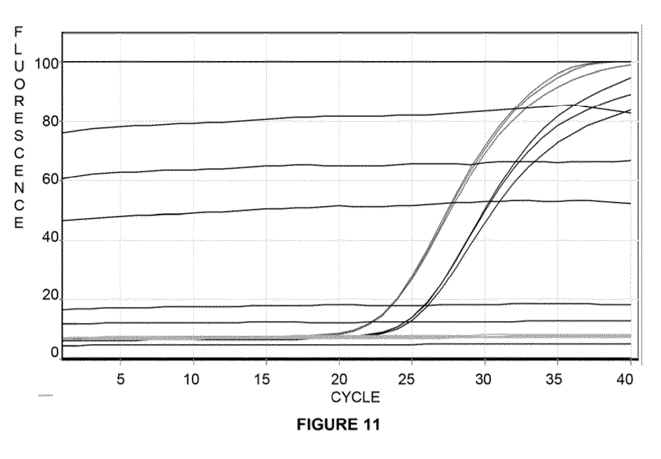

Hsa-miR-34a was amplified from rat brain RNA using a miRNA kit that utilises a FAM-tagged hydrolysis probe (Applied Biosystems). Following reverse transcription, the RNA was diluted one in three and a half, and both the neat (dark grey in FIG. 11) and the diluted (black in FIG. 11) cDNA were amplified in triplicate, along with a no-RT control (input RNA; light grey in FIG. 11) and a NTC (no cDNA, but all other reagents: light grey in FIG. 11). FAM-tagged reference nucleic acid (AccuCal-P) was diluted to 0 nM, 20 nM, 40 nM, 60 nM, B0 nM, 100 nM, 120 nM, 140 nM, 160 nM and 200 nM and used to determine the amount of input nucleic acid (Table 11).

TABLE 11

| | Mean calculated |
|---|---|
| Sample | amount (c/PCR) |
| Sample 1 | 4.42 × 10⁵ |
| Sample 2 | 1.32 × 10⁵ |
| No RT | 0 |
| NTC | 0 |

Although the invention has been described by way of examples, it should be appreciated that variations and modifications may be made without departing from the scope of the invention. Furthermore, where known equivalents exist to specific features, such equivalents are incorporated as if specifically referred to in this specification.

1. A method for quantifying a target nucleic acid, the method comprising the steps of:
(a) measuring the fluorescence of known quantities of a fluorophore-tagged universal reference nucleic acid to generate a calibration curve;
(b) amplifying the target nucleic acid in the presence of a fluorophore-tagged probe, wherein the fluorophore-tagged probe hybridizes with the target nucleic acid;
(c) measuring fluorescence during amplification of the target nucleic acid;
(d) comparing fluorescence during amplification with the calibration curve and determining the quantity of the amplified target nucleic acid.

2. The method of claim 1, wherein steps (a) to (d) are conducted substantially simultaneously.

3. The method of claim 1, wherein the fluorophore-tagged universal reference nucleic acid is single stranded.

4. The method of claim 1, wherein the calibration curve is generated using from about 0 ng to about 500 nM of fluorophore-tagged universal reference nucleic acid.

5. The method of claim 4, wherein the calibration curve is generated using from about 0 ng to about 200 nM of fluorophore-tagged universal reference nucleic acid.

6. The method of claim 1, wherein the calibration curve is generated using at least three known quantities of the fluorophore-tagged universal reference nucleic acid.

7. The method of claim 1, wherein amplification of the target nucleic acid is by polymerase chain reaction (PCR).

8. The method of claim 7, wherein the PCR is real-time PCR.

9. The method of claim 8, wherein the real-time PCR is multiplex PCR.

10. The method of claim 1, wherein the fluorophore-tagged universal reference nucleic acid and the fluorophore-tagged probe are detectable in the same emission channel.

11. The method of claim 7, wherein the PCR is performed over about 30 to about 45 cycles.

12. The method of claim 1, wherein the fluorophore-tagged universal reference nucleic acid and the fluorophore-tagged probe have the same excitation and emission spectra.

13. A kit comprising:
(a) one or more fluorophore-tagged universal reference nucleic acids;
(b) one or more fluorophore-tagged probes; and
(c) instructions for use in the method of claim 1.

14. A kit when used in the method of claim 1, the kit comprising:
(a) one or more fluorophore-tagged universal reference nucleic acids; and
(b) one or more fluorophore-tagged probes.
